(19) United States
(12) Patent Application Publication
(10) Pub. No.: US 2022/0175266 A1
(43) Pub. Date: Jun. 9, 2022

(54) INDUCTIVE PLETHYSMOGRAPHY VEST FOR A SMALL MAMMAL AND METHOD FOR MANUFACTURING SUCH A VEST

(71) Applicants: ETISENSE, LYON (FR); UNIVERSITE GRENOBLE ALPES, SAINT MARTIN D'HERES (FR)

(72) Inventors: Charles EYNARD, LYON (FR); Timothé FLENET, LYON (FR); Julie FONTECAVE-JALLON, CORENC (FR); Stéphane TANGUY, MONTBONNOT-SAINT-MARTIN (FR)

(21) Appl. No.: 17/441,540

(22) PCT Filed: Apr. 6, 2020
(86) PCT No.: PCT/EP2020/059799

§ 371 Date: Sep. 21, 2021

(30) Foreign Application Priority Data
Apr. 5, 2019 (FR) FR1903671

(51) Int. Cl. A61B 5/0535; A61B 5/00

(52) U.S. Cl. CPC A61B 5/0535; A61B 5/6805; A61B 2562/12; A61B 2503/42; A61B 2503/40

(57) ABSTRACT

The present invention relates to an inductive plethysmography vest for a mammal having a mass less than 6 kg, comprising:
a tube (10) made of elastic textile configured to surround the trunk of said mammal, said tube (10) extending along a longitudinal axis (X) parallel to the spinal column of said mammal;
at least one inductive winding formed of a strand (20) of electrically conductive wire arranged in the form of wavelets along the circumference of said tube (10);
said vest being characterised in that it comprises at least one strip (11) made of elastic textile, arranged along the circumference of the tube, defining a plurality of longitudinal loops (111), each wavelet being formed by the passage of the strand (20) of conductive wire successively in each loop (111).

### FIELD OF THE INVENTION

The invention relates to an inductive plethysmography vest for a small mammal and a method for manufacturing such a vest.

### PRIOR ART

Inductive plethysmography is a tool for measuring variations in volume of the trunk of a mammal (human or animal), for respiratory and/or cardiac applications.

The principle of this measurement is to wind around the trunk of the subject at least one inductive winding formed of one or more strands of electrically conductive wire. A variation in volume of the trunk, due notably to respiratory and/or cardiac activity, leads to a variation in inductance of each winding, which is measured by a sensor.

The reliability of the measurements necessitates ensuring that the position of the winding(s) with respect to the subject is constant over time. In particular, it is necessary to avoid any sliding of each winding vis-à-vis the trunk of the subject. Furthermore, in the case of a plurality of successive recordings, it is necessary to position each winding at the same spot on the trunk of the subject to ensure reproducibility of the measurements.

For human beings, it is known to sew each winding onto a band of an elastic textile and to wind said band around the trunk of the subject. In an alternative manner, each winding or band may be sewn onto a garment, such as a T-shirt, configured to be slipped onto the trunk of the subject with a slight tightening, which facilitates the putting in place of the plethysmography device. Reference could be made in this respect to the document U.S. Pat. No. 6,341,504.

For animals of medium size (such as dogs, primates, pigs), it is also known to use such bands, which are maintained on the animal by being integrated in a garment by push studs. The document U.S. Pat. No. 7,762,953 describes such a device.

These bands or garments require precise tailoring in order to procure correct positioning of the windings and to enable good reproducibility of the measurements over time and from one device to another. The fixation of the windings must further not lead to a stiffening of the vest in order not to prevent a variation in section of the windings and to minimise stress on the trunk of the subject.

For humans, for whom the device must be reusable, the sewing is generally carried out manually, which induces a high manufacturing cost or by knitting techniques limited to the production of elastic bands being able thereafter to be integrated in a garment, as described in the aforementioned document U.S. Pat. No. 6,341,504.

However, for small laboratory mammals, such tailoring poses three main problems. “Small mammal” is taken to mean in the present text the mammals normally used in laboratories and of which the mass is less than 6 kg. These mammals comprise in particular rodents (mice, rats, guinea pigs, hamsters, gerbils, ferrets) and lagomorphs (rabbits).

Firstly, the size of the windings is so reduced compared to that of a human or an animal of medium size that it makes manual sewing difficult. Secondly, it is difficult to knit and assemble elastic bands of small size on such a device without stiffening it. Thirdly, the device is configured to be worn by mammals of which the behaviours are of a nature to degrade it (perforations, tears, pull-offs, soiling). Its regular replacement being inevitable, its cost must thus be minimised.

For example, the document WO 2017/037369 describes an inductive plethysmography device for non-invasive measurement of aortic flow in the subdiaphragmatic region of a small laboratory mammal.

As illustrated in FIG. 1, the small mammal A is equipped with a thorax inductive plethysmography (TIP) vest 1 comprising two electrically conductive extensible windings 2, 3 integral with a tube made of elastic textile adjustable to the trunk of said mammal. In an optional manner, the vest 1 may further comprise an additional winding 5 arranged in the abdominal region to enable a respiratory inductive plethysmography (RIP) measurement.

The windings 2 and 3 are connected to an acquisition device 4 which makes it possible to record the signal of variation in section of each winding.

The windings 2 and 3 are shaped into wavelets to accompany the deformations of the sections that they surround and are positioned around two regions of the thorax of the animal.

The positioning and the configuration of the windings (amplitude and spatial period of the wavelets) are determined by a functional physiological model on which is based the calculation of the subdiaphragmatic aortic flow and standardised according to morphological criteria (weight ranges, species concerned). For small mammals, the diameter of the windings in the free state is typically comprised between 2 and 15 cm. Furthermore, the space between two windings is typically comprised between 0.5 and 5 cm. As regards the amplitude and the spatial period of the wavelets of the windings, they are respectively of the order of 0.5 to 5 cm and 0.5 to 3 cm.

The device further comprises a processor configured to calculate the instantaneous subdiaphragmatic aortic flow of the small mammal, from said signals of variation in section of each winding and from a functional model of the cardio-respiratory system according to which exchanges of blood between the thorax and the remainder of the body of said mammal consist in an output of blood via the abdominal aorta in the subdiaphragmatic region and in an input of blood via the inferior vena cava.

No method exists today making it possible to manufacture the vest with a sufficiently precise quality and a satisfactory cost.

### DESCRIPTION OF THE INVENTION

An aim of the invention is thus to design an inductive plethysmography vest for small laboratory mammal that has good reproducibility of the position and the size of the windings and which is inexpensive to manufacture.

To this end, the invention proposes an inductive plethysmography vest for a small mammal, that is to say a mammal having a mass less than 6 kg, comprising:
a tube made of elastic textile configured to surround the trunk of said mammal, said tube extending along a longitudinal axis parallel to the spinal column of said mammal;
at least one inductive winding formed of a strand of electrically conductive wire arranged in the form of wavelets along the circumference of said tube;

said vest being characterised in that it comprises at least one strip made of elastic textile, arranged along the circumference of the tube, defining a plurality of longitudinal loops, each wavelet being formed by the passage of the strand of conductive wire successively in each loop.

The circumference of the tube extends around the longitudinal axis of the tube. In other words, when the tube is presented flat, its circumference is perpendicular to the longitudinal axis.

Furthermore, the strip made of elastic textile is distinct from the tube and assembled thereon, by means detailed below.

Thus, each loop presents itself as a ring delimited on the one hand by the tube and on the other hand by the strip, extending in the longitudinal direction of the tube, and having two opposite ends in said longitudinal direction, suitable for passing a strand of conductive wire inside said ring, to maintain it between said ends, in the manner of the loop of a belt.

The loops advantageously constitute the sole means of maintaining the shape of the wavelets, and notably make it possible to do without any other means of fixation of the strand of conductive wire on the tube, such as sewing, knitting, bonding, etc.

In particular, the loops procure a sliding connection between the strand of conductive wire and the tube, unlike the previously cited techniques which form a rigid connection between the strand of conductive wire and the tube. This connection has several advantages. On the one hand, it makes it possible to minimise stiffening of the vest thanks to the possibility for the strand of wire to slide inside the loops when the tube is deformed. On the other hand, it enables easy replacement of the strand of conductive wire, for example in the event of damage thereto.

In a particularly advantageous manner, each loop is delimited by two longitudinal lines for assembling the strip on the tube.

Preferably, the distance between two adjacent assembly lines is constant.

According to an embodiment, the tube further comprises two orifices suitable for the passage of the front legs of the mammal, each orifice having an elongated shape along the circumference of the tube.

In an advantageous manner, the tube further comprises a reinforcement formed by a flap of the tube on the side of the front legs of the mammal, said orifices extending through said flap.

According to an embodiment, the vest further comprises a housing for an acquisition device connected to each inductive winding, said housing being formed in one piece with the tube or directly made integral with the tube.

According to another embodiment, the vest further comprises a harness comprising a housing for an acquisition device connected to each inductive winding and a strap suitable for maintaining the tube around the abdomen of the mammal.

Said strap may be provided with a plurality of gripping parts of hook and loop type.

According to an embodiment, each strand of electrically conductive wire passes at least twice in each loop along the circumference of the tube, so as to form a set of windings constituting a coil.

Another subject matter of the invention relates to a method for manufacturing such a vest. Said method comprises:
providing a band made of elastic textile having a longitudinal axis;
assembling on the band at least one strip made of elastic textile perpendicular to the longitudinal axis, along a plurality of lines parallel to said longitudinal axis, so as to define a plurality of loops between two adjacent lines of said strip;
closing the band along a line parallel to the longitudinal axis so as to form a tube;
forming at least one inductive winding along the circumference of the tube by passing a strand of electrically conductive wire successively in each loop of a respective strip so as to form wavelets.

The assembly of the strip on the tube may advantageously be carried out by ultrasonic welding, sewing and/or bonding.

According to an embodiment, the method further comprises piercing in the band two orifices suitable for the passage of the front legs of the small mammal by means of a punch.

The method may further comprise, before the piercing of said orifices, assembling a flap of the band on one face of the tube, the orifices being pierced through said flap.

The invention also relates to a method for manufacturing a set of inductive plethysmography vests for small mammals of different sizes, the mass of said mammals being comprised between 20 and 6000 g, preferably between 100 and 6000 g, wherein the method is implemented such as described above for each of said vests, the distance between two adjacent assembly lines of each strip being identical for all of the vests.

### BRIEF DESCRIPTION OF THE FIGURES

Other characteristics and advantages of the invention will become clear from the detailed description that follows, with reference to the appended drawings in which:

FIG. 1 is a schematic view of a rat wearing an inductive plethysmography vest such as described in the document WO 2017/037369;

FIG. 2 is a view of the pattern of the vest (on the side of its inner face) according to an embodiment;

FIG. 3 is a view of the pattern of FIG. 2, on the side of the outer face of the vest;

FIG. 4 is a view of the assembled band;

FIG. 5 is a view of the assembled band with multiple inductive windings;

FIG. 6 is a view of the pattern of the harness (on the side of its outer face) according to an embodiment;

FIG. 7 is a view of the pattern of FIG. 6, on the side of the inner face of the harness;

FIG. 8 is a view of the vest assembled after closing of the band on itself to form the tube;

FIG. 9 is a view in perspective of a vest assembled according to another embodiment,

FIG. 10 is a view of the pattern of the vest of FIG. 9.

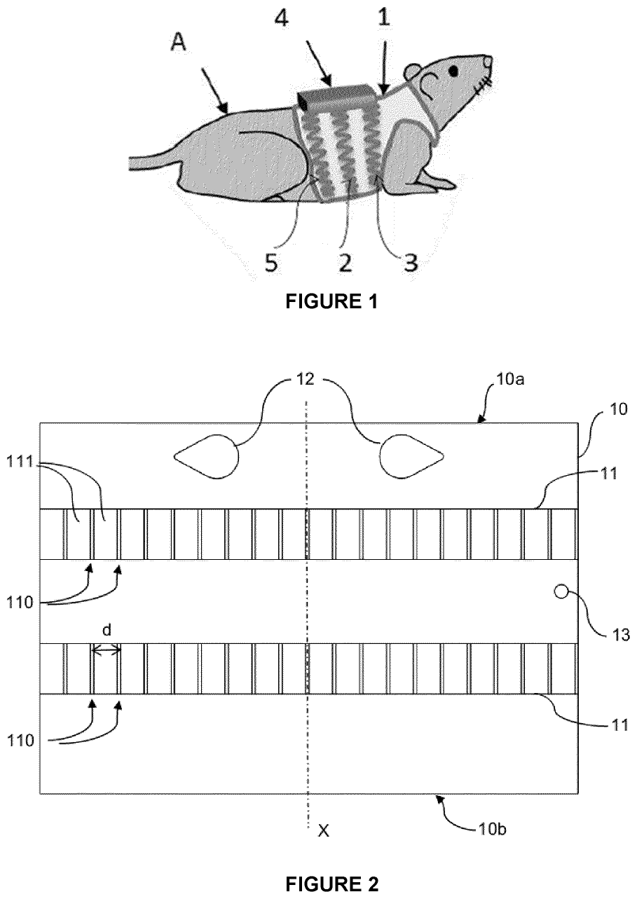

Identical reference signs from one figure to the other designate identical elements or elements fulfilling the same function.

### DETAILED DESCRIPTION OF EMBODIMENTS

According to the invention, the inductive plethysmography vest for a small mammal comprises a tube made of elastic textile configured to surround the trunk of said mammal.

The tube comprises a longitudinal axis which is substantially parallel to the spinal column of the animal.

The tube may have a longitudinal closing, for example arranged along the spinal column of the animal, in order to facilitate the putting in place of the tube on the trunk. Such a closing may comprise fastening elements of hook and loop type (better known by the name VELCRO™), push studs, a slide fastener, etc.

Alternatively, the tube is closed on itself in a permanent manner, by welding, bonding or sewing for example; it must then be slipped onto the trunk of the animal, the elasticity of the textile being chosen to enable such a slipping on with adjustment to the trunk of the animal.

The tube serves as support for at least one inductive winding formed of a strand of electrically conductive wire arranged in the form of wavelets along the circumference of said tube.

To this end, the vest comprises at least one strip made of elastic textile arranged on the circumference of the tube and defining a plurality of longitudinal loops, each wavelet being formed by the passage of the strand of conductive wire successively in each loop.

The strip is assembled on the tube along longitudinal lines, such that two adjacent lines define a loop. The assembly may be carried out by welding, by sewing, by bonding, or by any other suitable means.

Welding (notably ultrasonic welding) is particularly preferred in so far as it may be carried out using a device of reduced bulk, on the outer side of the strip (which is easily accessible); in addition, this welding may be implemented by means of a template serving to guide the welding along the longitudinal lines and making it possible to define the width of the loops.

Being carried out in the longitudinal direction, this assembly does not affect the elasticity of the tube and each strip in the circumferential direction. Thus, it does not impede the deformation of the winding(s) during the activity of the animal and does not disrupt the plethysmographic measurements. Even if the assembly is capable of stiffening the tube in the longitudinal direction, such a stiffening is not penalising for the measurements.

The width of the loops (distance between two adjacent lines) defines the period of each winding. This width is typically chosen to adjust the number of wavelets between a minimum value (for example 10) making it possible to guarantee the elasticity of each winding and a maximum value (for example 20) making it possible to limit the time required to put the wire in place in all of the loops. In practice, a width of the order of 4 to 40 mm is preferred. The aforementioned template makes it possible to ensure a constant period of the windings over the whole circumference of the tube.

In a particularly advantageous manner, this width of the loops may even be constant between vests of different sizes. Indeed, for small mammals of which the mass varies between 20 g and 6 kg, it is possible to define a limited number of vests of different sizes, each suitable for a given range of small mammals. The conservation of the width of the loops from one size to the other makes it possible to simplify the manufacture of the vest and to conserve the same period for each winding.

According to other embodiments, the vest may comprise two or more inductive windings, arranged at different spots of the thorax and/or the abdomen of the animal. In this case, the vest comprises as many strips as windings, said strips being arranged parallel with each other over the length of the tube, each strip defining a plurality of longitudinal loops in which each electrically conductive wire is arranged to form wavelets.

According to an advantageous embodiment, each strand of electrically conductive wire passes at least twice in the loops of a same strip along the circumference of the tube, so as to form a set of windings constituting a coil. This makes it possible to increase the sensitivity of the windings. The self-inductance L (in henry (H)) of a coil in air is in fact defined by the formula:

$$L = \frac{{μ}_{0} {N}^{2} S}{l}$$

where μ₀ is the magnetic constant, equal to 4π*10⁻⁷ H·m⁻¹, N is the number of windings, S is the section of the coil in m², and l is the length of the coil in m.

The self-inductance value is multiplied by a factor N²/l for a same surrounded section. Compared to a single winding, a coil constituted of N windings necessitates a wire N times longer. The self-inductance of each sensor (L_{N}) is thus theoretically equal to the self-inductance of a winding (L_{N=1}) multiplied by a factor N.

In an advantageous manner, the vest comprises two orifices for the passage of the front legs of the animal. In so far as the section of the legs is not circular, said orifices are preferably non-circular. Preferably, said orifices have an elongated shape (for example a drop of water shape) in the circumferential direction of the tube. Thus, the vest can be adjusted more closely to the trunk of the animal in the region of the front legs. This shape is better suited to the morphology of the limbs of the animal; it prevents discomfort linked to the wearing of the vest and ensures better maintaining over time.

The front part of the tube (situated on the side of the front legs) may be stiffened by a reinforcement. In a particularly advantageous manner, said reinforcement may be constituted by a flap of the tube on the outer side thereof, assembled on the outer face of the tube by one of the assembly methods mentioned above. Preferably, this reinforcement extends at least up to the rear of the front legs of the animal; thus, the orifices for the front legs extend through a double thickness of elastic textile, which contributes to reinforcing the mechanical strength of the tube in this region.

According to an embodiment, the vest further comprises a harness configured to support a connector electrically connected to the electrically conductive wire and/or to increase the maintaining of the tube on the trunk of the animal.

This harness advantageously comprises a pocket suitable for receiving a connector suitable for electrically connecting the electrically conductive wire(s) and the plethysmographic measurement chain. Said pocket is configured to be positioned on the back of the animal.

In an alternative manner, the pocket may be formed directly on the outer face of the tube.

The vest described above may be manufactured in a simple manner while ensuring good precision and reproducibility of the positioning of the windings.

The tube is formed from a band made of elastic textile which is closed on itself along a line parallel to the longitudinal axis of the tube.

The closing may be permanent, that is to say non-dismountable without damaging the vest, for example by welding of the band on itself.

In an alternative manner, the closing may be reversible, for example thanks to gripping parts of hook and loop type, buttons, etc.

Before being closed on itself, the band is assembled with at least one strip along the longitudinal lines which delimit the loops in said strip. As indicated above, this assembly may be carried out by welding (notably ultrasonic welding), bonding, sewing, etc. The advantage of this operation is that it is carried out flat, a template being able to be used to guarantee a constant distance between the assembly lines.

Before or after the closing of the tube on itself, an electrically conductive wire is threaded successively in all of the loops of a same strip. Once the tube has been closed, the wire thus forms an inductive winding. To facilitate the putting in place of the wire in the narrow loops, the wire is advantageously threaded into the eye of a needle and introduced into the loop by means of said needle.

Each end of the wire is next crimped in a lug and said lug is inserted into a connector which is positioned on the back of the animal in a pocket provided for this purpose. The connector is itself connected to the plethysmographic measurement chain.

FIGS. 2 to 7 illustrate different components of the vest.

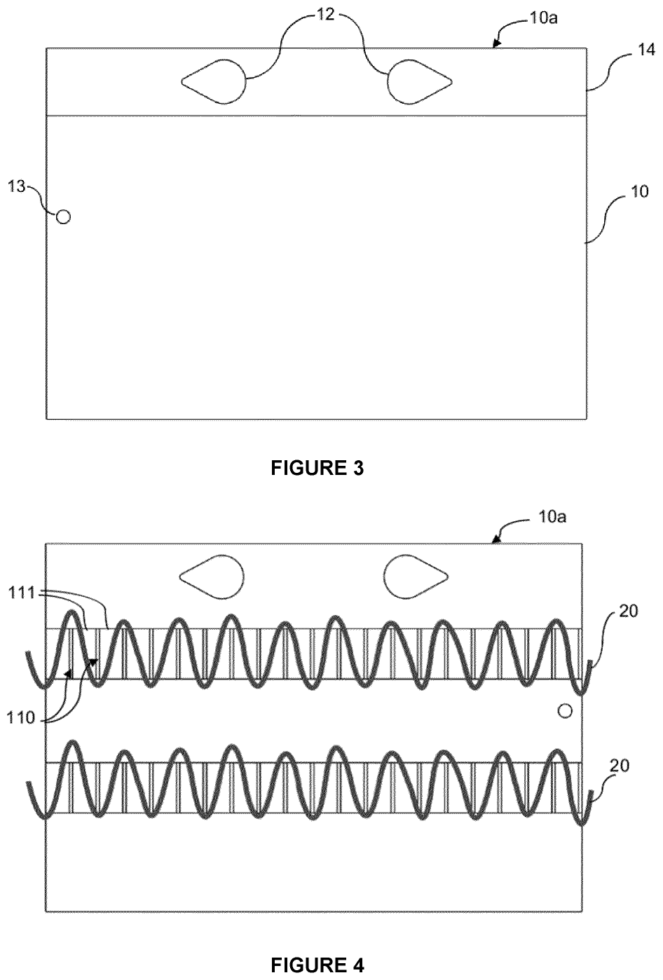

FIGS. 2 and 3 show the band of elastic textile configured to form the tube, FIG. 2 showing the inner face (configured to be in contact with the skin of the animal) and FIG. 3 the outer face (opposite to the skin of the animal).

The band 10 has a generally rectangular shape. 10a designates its anterior edge (situated on the side of the front legs of the animal) and 10b its posterior edge (situated on the side of the rear legs of the animal).

In an advantageous manner, the band is folded back on itself at the level of the anterior edge in order to form a flap 14 which is assembled on the outer face of the band (cf. FIG. 2). This flap makes it possible to reinforce the band, notably when orifices are pierced therein for the passage of the legs.

Two orifices 12 are formed in the front part of the band, preferably through the flap 14 if this is present. These orifices are for example cut out by means of a punch. In a particularly advantageous manner, the orifices 12 have an elongated shape in the circumferential direction. In the embodiment of FIGS. 2 and 3, each opening 12 has the shape of a drop of water, with a semi-circular portion on the inner side of each leg and a tapered portion on the outer side of each leg.

Two rectangular strips 11 are assembled on the inner face of the band 10 along the assembly lines 110. Said strips are perpendicular to the longitudinal axis X of the band, which is parallel to the spinal column of the animal. The lines 110 are parallel to the axis X. The distance d between two adjacent lines 110 is constant over the width of the band (corresponding to the circumference of the tube). Each pair of adjacent lines defines a respective loop 111.

One or more orifices 13, which may be circular, are arranged in the vicinity of the lateral edge of the band 10, in order to enable the passage of electrically conductive wires through the tube. Said orifices are for example cut out by means of a punch.

FIG. 4 represents the band after threading of a strand 20 of electrically conductive wire in the loops of each respective strip.

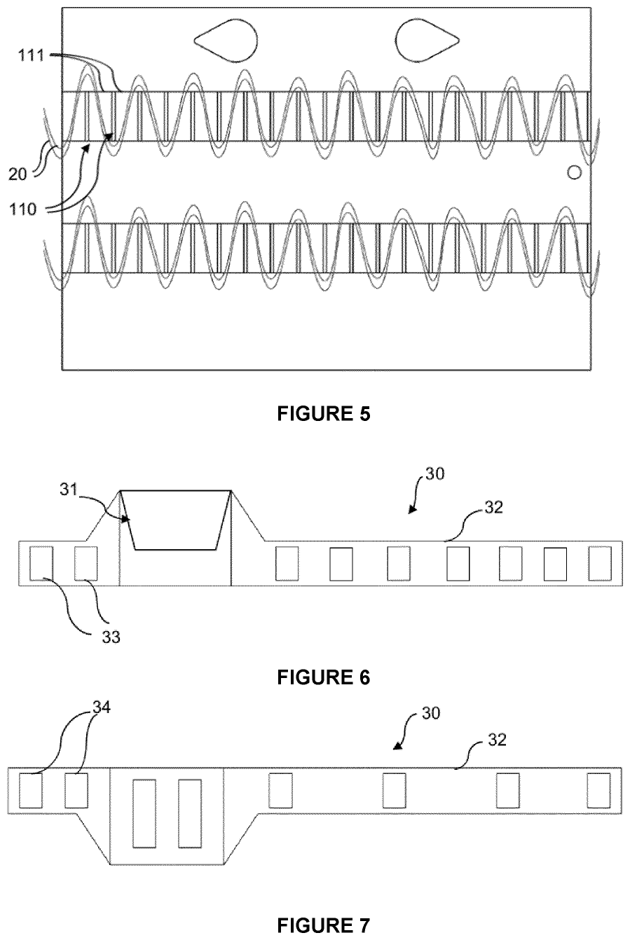

FIG. 5 represents an embodiment wherein two strands 20 of a same electrically conductive wire have been threaded in the loops of each respective strip to form, after closing of the tube, a coil with two windings.

FIGS. 6 and 7 respectively show the outer face and the inner face of an embodiment of the harness. The harness 30 comprises a pocket 31 which is formed for example by a flap of the extensible textile forming the harness. The inner dimensions of the pocket are adapted to that of the aforementioned connector. On either side of said pocket 31 extends a strap 32 of which the length is suitable for surrounding the trunk of the animal. The outer face and the inner face of the strap 32 comprise complementary fastening parts 33, 34, for example rectangles of VELCRO™ spread out over the length of the strap. Thus, whatever the section of the trunk of the animal, it is possible to juxtapose at least two complementary parts 33, 34 and thus adjust the strap to the trunk of the animal. The pocket and the strap are not necessarily made of one piece.

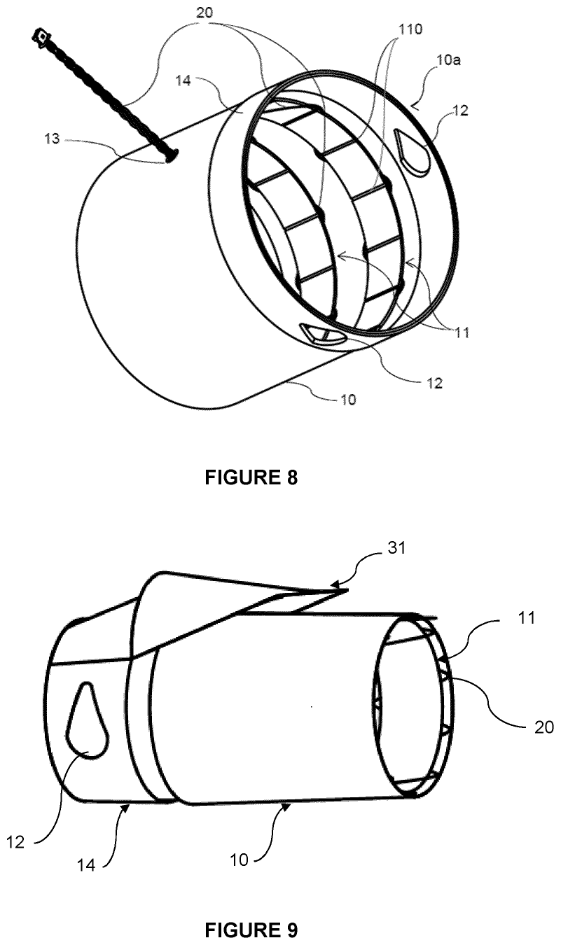

FIG. 8 represents the vest after closing of the band on itself to form a tube. The vest comprises two parallel strips 11, maintaining two electrically conductive windings each formed of a strand 20 of electrically conductive wire. The windings being formed on the inner side of the tube, the ends of the strands of electrically conductive wire emerge from the tube via the orifice 13 and are connected to a connector. The strands are advantageously twisted together.

FIGS. 9 and 10 illustrate another embodiment of the vest, in which the pocket is integrated in the vest, either in one piece with the vest, or by directly making integral with the vest. This embodiment is advantageous in that does away with the strap, which makes it possible to avoid exerting a too important compression of the abdomen of the animal. Furthermore, the integration of the pocket in the vest makes it possible to avoid any sliding of the pocket around the trunk of the animal and to ensure good centring of the acquisition device with respect to the back of the animal.

In certain embodiments, the pocket is formed by assembling two parallel tabs 14 formed in one piece with the band 10, at the level of the part configured to form the collar of the vest.

During the manufacture of the vest, the tabs are folded back on themselves towards the rear of the animal, and assembled by any suitable means (for example: bonding, welding, sewing, etc.) to form a pocket of size suited to the acquisition device. The opening of the pocket is situated at the level of the collar, so as to be inaccessible when the pocket is folded down on the back of the animal.

In other embodiments, the pocket is made integral with the tube by any suitable means (for example: bonding, welding, sewing, etc.), preferably at the level of the end forming the collar.

Furthermore, the band 10 may be lengthened rearwards in order to form a flap folded back on the back of the animal in order to cover the cables and electrodes of the acquisition device. Thus, the device is protected from projections of fluids or mechanical attacks notably when the animal is in the presence of congeners.

Applications

An application of the device described above is cardiac inductive plethysmography to implement for example the virtual aortic flow ring forming the subject matter of the document WO 2017/037369 according to different measurement configurations including, in a non-limiting manner:
a thoracic, abdominal or diaphragmatic band,
two thoracic bands,
two abdominal bands,
a thoracic band and a diaphragmatic band,
a diaphragmatic band and an abdominal band.

Another application of the device is respiratory inductive plethysmography with a model with one or more compartments of which the variations in section may be recorded by:
a thoracic or abdominal band,
an abdominal band and a thoracic band,
a thoracic band, an abdominal band and a diaphragmatic band.

Generally speaking, the device enables the monitoring of mechanical deformations of compartments with as many thoracic, diaphragmatic or abdominal bands as compartments to monitor.

The vest is to be made available in different models adapted as a function of the species and the weight of the animals, which is generally comprised between 20 and 6000 g, preferably between 100 and 6000 g. Relevant species notably comprise mice, rats, guinea pigs, rabbits, and juvenile animals such as small pigs or puppies.

For example, for an application in Wistar or Sprague Dawley strain rats, the range may be composed of 4 sizes of vests corresponding to the following weights:

| | Weight range |
|---|---|
| Size 0 | 100 to 200 g |
| Size 1 | 200 to 300 g |
| Size 2 | 300 to 400 g |
| Size 3 | Above 400 g |

The abdominal, thoracic and diaphragmatic positions are dependent on the morphology of the species and must be adapted as a consequence.

### REFERENCES

WO 2017/037369
U.S. Pat. No. 6,341,504
U.S. Pat. No. 7,762,953

1. An inductive plethysmography vest for a mammal having a mass less than 6 kg, comprising:
a tube made of elastic textile configured to surround the trunk of said mammal; said tube extending along a longitudinal axis parallel to a spinal column of said mammal;
at least one inductive winding formed of a strand of electrically conductive wire arranged in the form of wavelets along the circumference of said tube; and
at least one strip made of elastic textile, arranged along a circumference of the tube, defining a plurality of longitudinal loops, each wavelet being formed by the passage of the strand of conductive wire successively in each loop.

2. The vest of claim 1, wherein each loop is delimited by two longitudinal lines for assembling the strip on the tube.

3. The vest of claim 2, wherein a distance between two adjacent assembly lines is constant.

4. The vest of claim 1, wherein the tube further comprises two orifices suitable for the passage of the front legs of the mammal, each orifice having an elongated shape along the circumference of the tube.

5. The vest of claim 4, wherein the tube further comprises a reinforcement formed by a flap of the tube on the side of the front legs of the mammal, said orifices extending through said flap.

6. The vest of claim 1, further comprising a housing for an acquisition device connected to each inductive winding, said housing being formed in one piece with the tube or directly made integral with the tube.

7. The vest of claim 1, further comprising a harness comprising a housing for an acquisition device connected to each inductive winding and a strap suitable for maintaining the tube around an abdomen of the mammal.

8. The vest of claim 7, wherein the strap is provided with a plurality of gripping parts of hook and loop type.

9. The vest of claim 1, wherein each strand of electrically conductive wire passes at least twice in each loop along the circumference of the tube, so as to form a set of windings constituting a coil.

10. A method for manufacturing an inductive plethysmography vest for a mammal of mass less than 6 kg, comprising:
providing a band made of elastic textile having a longitudinal axis;
assembling on the band at least one strip made of elastic textile perpendicular to the longitudinal axis, along a plurality of lines parallel to said longitudinal axis, so as to define a plurality of loops between two adjacent lines of said strip;
closing the band along a line parallel to the longitudinal axis so as to form a tube; and
forming at least one inductive winding along a circumference of the tube by passing a strand of electrically conductive wire successively in each loop of a respective strip so as to form wavelets.

11. The method of claim 10, wherein the assembly of the strip on the tube is carried out by ultrasonic welding, sewing and/or bonding.

12. The method of claim 10, further comprising piercing in the band two orifices suitable for the passage of the front legs of the small mammal by means of a punch.

13. The method of claim 12, further comprising, before the piercing of said orifices, assembling a flap of the band on one face of the tube, the orifices being pierced through said flap.

14. A method for manufacturing a set of inductive plethysmography vests for small mammals of different sizes, the mass of said mammals being comprised between 20 and 6000 g, wherein the method is implemented according to claim 10 for each of said vests, a distance between two adjacent assembly lines of each strip being identical for all of the vests.
